(19) United States
(12) Patent Application Publication
(10) Pub. No.: US 2018/0263558 A1
(43) Pub. Date: Sep. 20, 2018

(54) A PHYSIOLOGICAL MONITORING KIT WITH USB DRIVE

(71) Applicant: KONINKLIJKE PHILIPS N.V., EINDHOVEN (NL)

(72) Inventors: John E. CRONIN, BONITA SPRINGS, FL (US); Steven PHILBIN, BONITA SPRINGS, FL (US)

(21) Appl. No.: 15/761,207

(22) PCT Filed: Sep. 27, 2016
(86) PCT No.: PCT/EP2016/072887

§ 371 Date: Mar. 19, 2018

Related U.S. Application Data
(60) Provisional application No. 62/233,578, filed on Sep. 28, 2015.

(30) Foreign Application Priority Data
Mar. 22, 2016 (EP) 16161499.5

(51) Int. Cl. A61B 5/00; G16H 40/63; A61B 5/021; G01K 13/00

(52) U.S. Cl. CPC A61B 5/4842; G16H 40/63; A61B 2560/029; G01K 13/002; A61B 5/002; A61B 5/021

(57) ABSTRACT

Systems and methods for monitoring a patient with a specific condition by using a kit with monitoring devices and a corresponding memory device for the specific condition. The system comprises a health monitor, and a plurality of monitoring devices for acquiring patient health data. The kit further comprises one or more monitoring devices—such as pulse oximeters, sphygmomanometers, thermometers, EEG and ECG leads, blood 5 glucose meters, and other health monitoring devices relating to the condition specific to the kit. Such a system may be useful as a means of identifying current patient condition and determining which steps to take in the emergency situation.

### BACKGROUND

Patient monitoring is an essential aspect of hospitals, emergency rooms, intensive care units, surgery rooms, nursing homes, and doctors' offices. It involves the use of health monitoring devices such as thermometers, sphygmomanometers, and pulse oximeters. However, there is a need for a better way to monitor patients with specific diseases or conditions. In this regard, a kit may be better suited to provide proper monitoring of patients with specific conditions. For example, the kit is especially convenient for use in emergency situations because medical professionals can simply select the kit needed in each situation which can save valuable time.

Memory devices may be integrated into a removable module, in which the processor executes a preloaded program to receive the physiological signals from the physiological signal sensing unit. Here, the memory device is capable of electrically connecting with a docking unit. The docking unit is driven by a processor for displaying the physiological information of the patient. The patent application also discloses an alarm signal when the physiological signals match a preset condition.

In the context of patient monitoring, a kit is useful to medical professionals for monitoring patient health data with the proper monitoring devices for the patient's specific condition. The kit may also be used in emergency situations to allow quick and easy access to diagnostic equipment and monitoring of patients.

### SUMMARY OF THE CLAIMED INVENTION

Embodiments of the present invention relates to systems and methods for monitoring a patient with a specific condition. The method according to some embodiments comprises providing a health monitor and at least one kit containing at least one monitoring device and a memory device. The memory device stores at least one condition-specific software, protocols, and alerts of the monitoring devices. The memory device may be connected to the health monitor. The protocols and alerts of the monitoring devices are uploaded in the health monitor protocols and alerts database. The health monitor accesses a health network via a cloud network to compare the stored protocols and alerts with the corresponding protocols and alerts in the health network database. A devices database of the health monitor is then accessed to determine monitoring devices contained in the kit. The protocols and alerts database of the health monitor is updated to match the database on the health network. Lastly, the condition-specific software is executed to receive the readings from the monitoring devices and store the received readings in the sensor database of the health monitor.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system according to a preferred embodiment of the present invention.

FIG. 2 illustrates a flowchart for carrying out a preferred embodiment of the invention.

FIG. 3 illustrates a flowchart of the software for receiving the readings from the monitoring devices.

FIG. 4 illustrates a flowchart of the software for updating monitoring protocols and devices based on cloud data.

FIG. 5 illustrates an exemplary user interface of the health monitor in accordance with an embodiment of the invention.

FIG. 6 illustrates an exemplary user interface of the health monitor in accordance with an embodiment of the invention.

FIG. 7 illustrates an exemplary system in accordance with an embodiment of the invention.

FIG. 8A and FIG. 8B illustrate exemplary databases stored in the health monitor in accordance with an embodiment of the invention.

FIG. 9 illustrates an exemplary sensor database containing data from the health monitor in accordance with an embodiment of the invention.

### DETAILED DESCRIPTION

Some embodiments of the present invention relates to a method for monitoring a patient with a specific condition comprising: providing a health monitor and an at least one kit, wherein the at least one kit comprises: one or more monitoring devices for acquiring patient health data; a memory device comprising a means to connect the memory device to the health monitor and at least one condition-specific software comprising health-monitoring protocols and alerts for the acquired patient health data; connecting the memory device to the health monitor; accessing a health network via a network and comparing the health-monitoring protocols and alerts with the database on the health network corresponding to the at least one kit; accessing a health monitor devices database and determining monitoring devices for the at least one kit; updating the health monitor protocols and alerts database to match the database on the health network; and running the at least one condition-specific software.

The method for monitoring a patient with a specific condition is done by connecting a memory device to a port on the health monitor. The health monitor polls for a memory device with a specific-condition software. Data from the memory device is uploaded and stored into the corresponding devices database and protocols and alerts database of the health monitor. The health monitor accesses a health network via the cloud network. A comparison is made between the contents of the protocols and alerts database found in the health monitor and the contents in the health network database. If a difference is detected, the protocols and alerts are updated to match the corresponding data on the health network databases. The health monitor then polls for an at least one connected monitoring device. If the at least one monitoring device is connected, the monitoring software is executed.

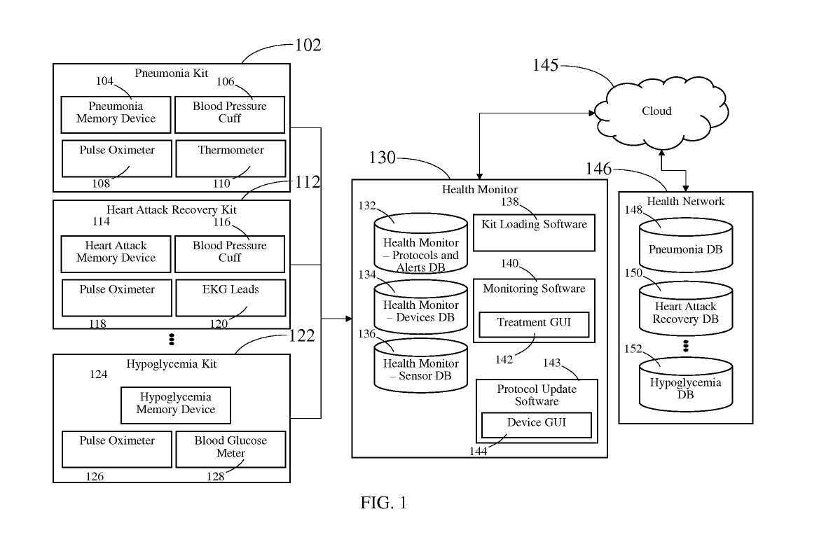

FIG. 1 illustrates a system according to a preferred embodiment of the present invention. The system comprises at least one kit (e.g., 102, 112, 122) containing an at least one monitoring device (e.g., 1006, 108, 110, 116, 118, 120, 126, and 128) and a memory device (e.g., 104, 114, 124). In an n example as shown in FIG. 1, the system for monitoring a patient with a specific condition comprises a pneumonia kit 102 which contains a pneumonia memory device 104, a blood pressure cuff 106, a pulse oximeter 108 and a thermometer 110. In another embodiment, the system comprises a heart attack recovery kit 112 which contains a heart attack memory device 114, a blood pressure cuff 116, a pulse oximeter 118 and EKG leads 120. In other embodiments, the system comprises a hypoglycemia kit 122 that contains a hypoglycemia memory device 124, a pulse oximeter 126 and a blood glucose meter 128. The system may also be used with other kits for various types of disease states.

In one embodiment of the present invention, the memory device comprises a means to connect the memory device to the health monitor and a software for a specific disease state. Preferably, the memory device stores protocols and alerts used for the acquisition of patient health data. The memory device also stores a list of devices contained in the at least one kit.

As shown in FIG. 1, the system also comprises a health monitor 130 which further comprises a protocols and alerts database 132, a devices database 134, a sensor database 136, a kit loading software 138, a monitoring software 140 with a treatment GUI 142, and a protocol update software 143 with a device GUI 144. The system further comprises a health network 146 accessed via a cloud network 145. The health network further comprises an at least one disease database such as a pneumonia database 148, a heart attack recovery database 150, a hypoglycemia database 152 and other databases for various types of diseases.

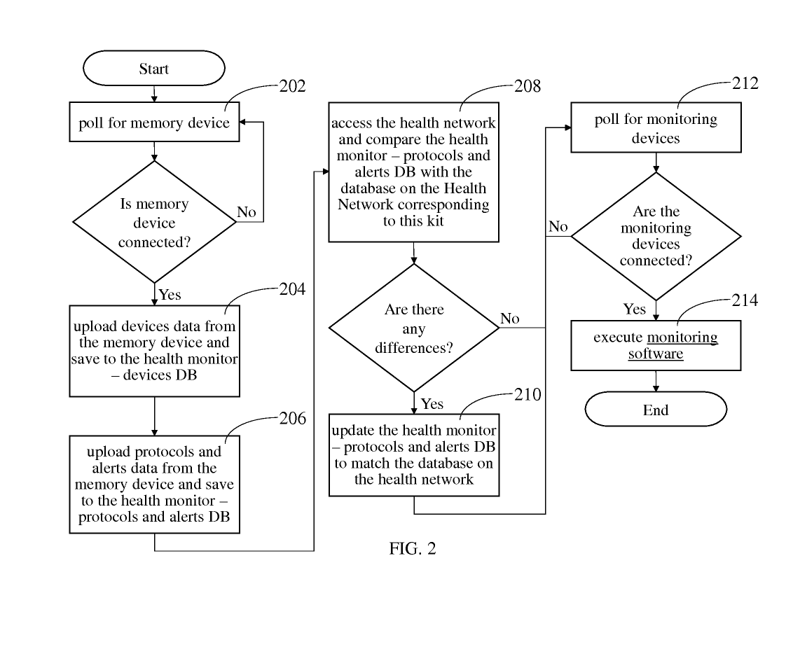

FIG. 2 shows a flowchart illustrating a preferred method of the present invention. A health monitor 130 polls for a connected memory device (step 202). If there is no memory device present, the health monitor loops until a connection is detected. Upon the detection of the memory device, the list of monitoring devices contained in the at least one kit are uploaded and stored to the devices database 134 on the health monitor 130 (step 204). Also, the protocols and alerts data (i.e., one or more thresholds for the acquired patient health data) from the memory device are uploaded and stored into the protocols and alerts database 132 on the health monitor 130 (step 206). Subsequently, the health network 146 is accessed via the cloud network 145 by the health monitor 130 (step 208). The protocols and alerts database 132 on the health monitor 130 is compared with the corresponding disease databases on the health network 146. If there are differences detected, the protocols and alerts database 132 on the health monitor 130 are updated to match the corresponding data in the disease databases on the health network 146 (step 210). Next, the health monitor 130 polls for an at least one connected monitoring device (step 212). If an at least one monitoring device is detected, the monitoring software 140 is executed (step 214).

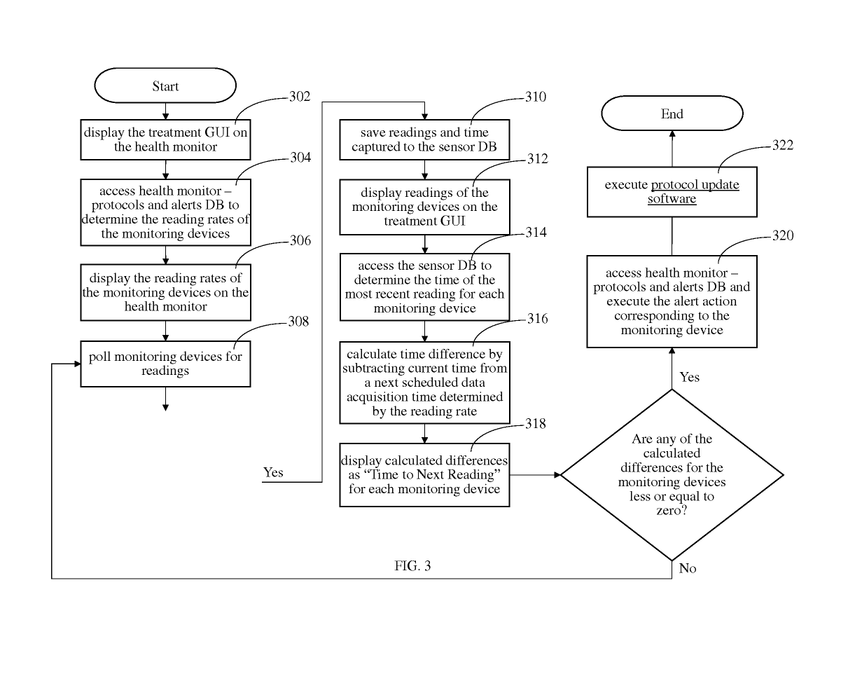

FIG. 3 shows a flowchart of the monitoring software 140 described previously. The monitoring software 140 undergoes initialization only when it detects at least one connected monitoring device. A treatment GUI 142 is displayed on the health monitor 130 (step 302). The protocols and alerts database 132 in the health monitor 130 is accessed to determine the reading rates of the at least one monitoring device (step 304). The reading rates are displayed on the treatment GUI 142 (step 306). The monitoring software 140 then polls the monitoring devices for readings (step 308). The detected readings from the monitoring devices are stored into the sensor database 136 on the health monitor 130 (step 310). The readings are then displayed onto the treatment GUI 142 of the health monitor 130 (step 312). Thereafter, the sensor database 136 is accessed to determine the most recent readings for each of the at least one monitoring device (step 314). Next, a time difference is calculated by subtracting the current time from a next scheduled data acquisition time determined by the reading rate of the monitoring device (step 316). Thereafter, the calculated difference is displayed on the treatment GUI 142 (step 318) and labeled as “Time to Next Reading.” The protocols and alerts database 132 is accessed and alert actions corresponding to the monitoring devices are executed (step 320). The protocol update software 143 is executed (step 322).

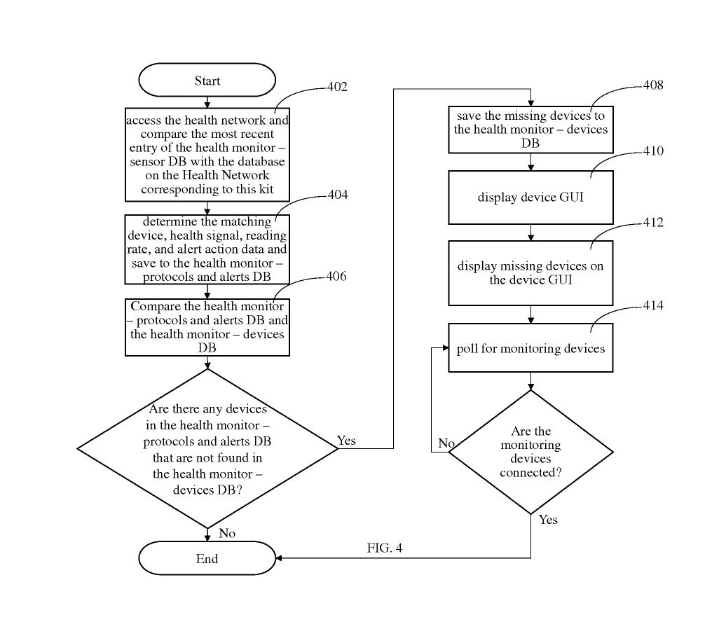

FIG. 4 shows a flow chart of the protocol update software 143 described previously. The health network 146 is accessed and the disease database on the Health Network corresponding to the kit being used is compared with the most recent entry of the health monitor—sensor DB 136 (step 402). The matching device, health signal, reading rate, and alert action data on the corresponding health network database is determined and saved to the health monitor—protocols and alerts DB 132 (step 404). The health monitor—protocols and alerts DB 132 is compared with the health monitor—devices DB 134 to determine if there are any devices in the health monitor protocols and alerts DB 132 that are not found (missing) in the health monitor devices DB 134 (step 406). The determined missing devices are saved to the health monitor devices DB (step 408). The device GUI 144 is displayed on the health monitor 130 (step 410). The missing devices previously determined by step 406 are displayed on the device GUI 144 (step 412). Next the protocol update software 143 polls for all the monitoring devices of the health monitor devices DB 134 being connected to the health monitor 130 (step 414). If they are all connected, the protocol update software 143 finishes its execution.

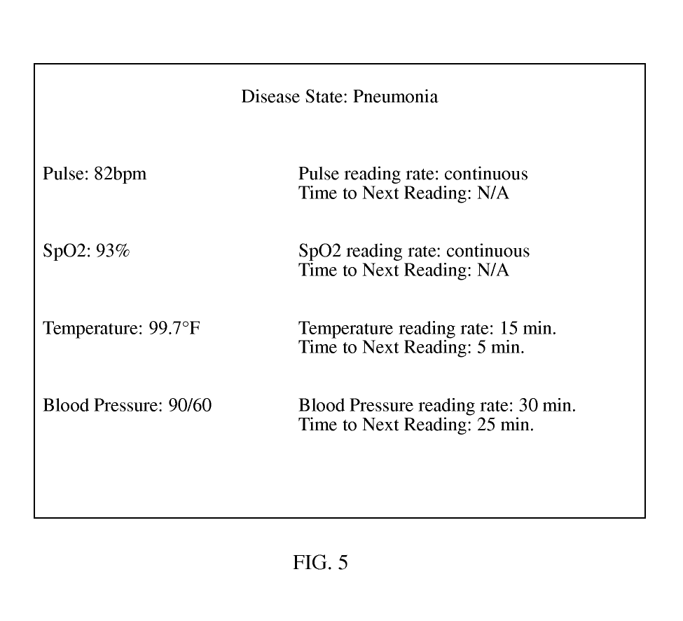

FIG. 5 shows an exemplary model of the treatment GUI 142 of the monitoring software 140. The display shows the disease state being monitored, the relevant sensors (monitoring devices) connected (acquiring the patient health data)and their corresponding readings of the patient health data, the corresponding reading rates of each sensors and the “Time to Next Reading” calculated from the monitoring software 140.

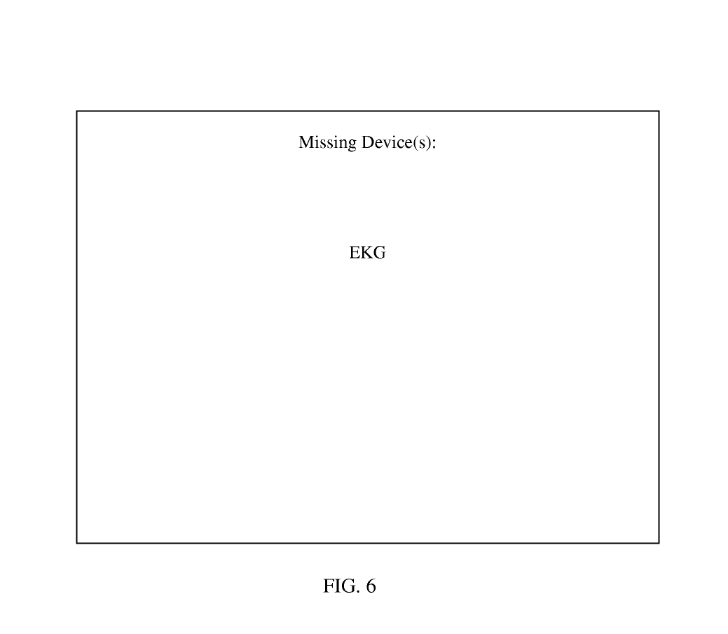

FIG. 6 shows an exemplary model of the device GUI 144 of the protocol update software 143. The display shows the missing device or devices as determined by the protocol update software.

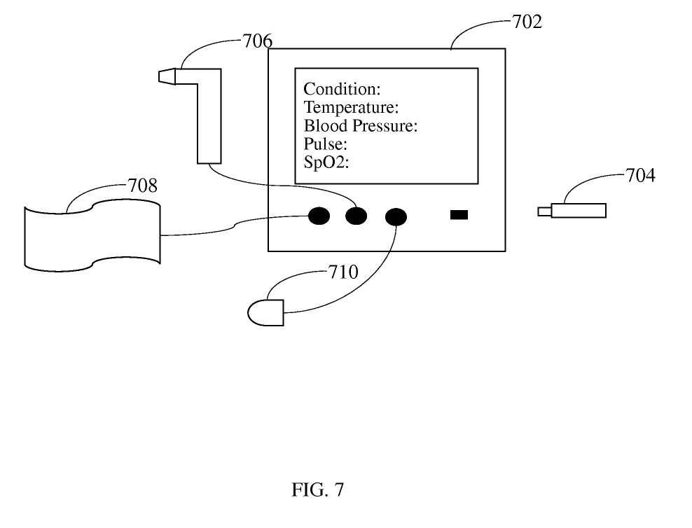

FIG. 7 shows an exemplary model of the system which comprises a health monitor 702. The health monitor has a plurality of ports for the memory device 704 and the monitoring devices 706-710. The monitoring devices, in the example shown in FIG. 7, are a thermometer 706, a blood pressure cuff 708 and a pulse oximeter 710.

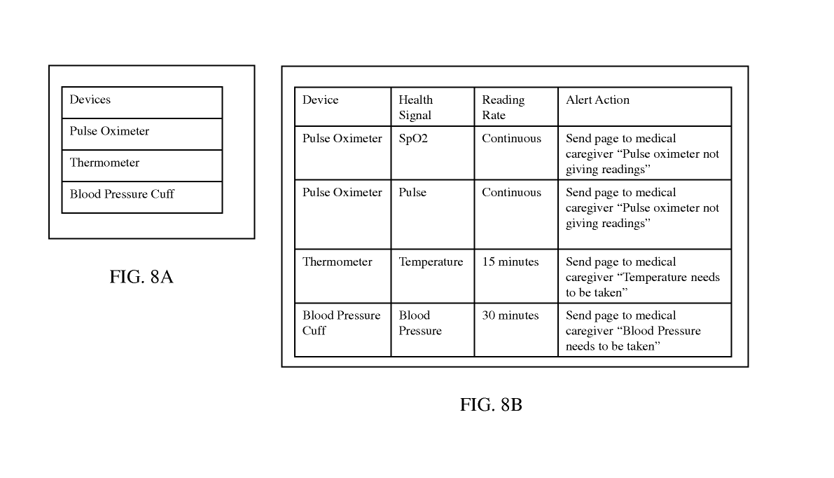

FIG. 8A shows an exemplary device database 134. The device database 134 illustrates the monitoring devices contained in the at least one kit. FIG. 8B shows exemplary protocols and alerts database 132 which include the monitoring device, the health signal or data being monitored, the reading rate, and the corresponding alert action.

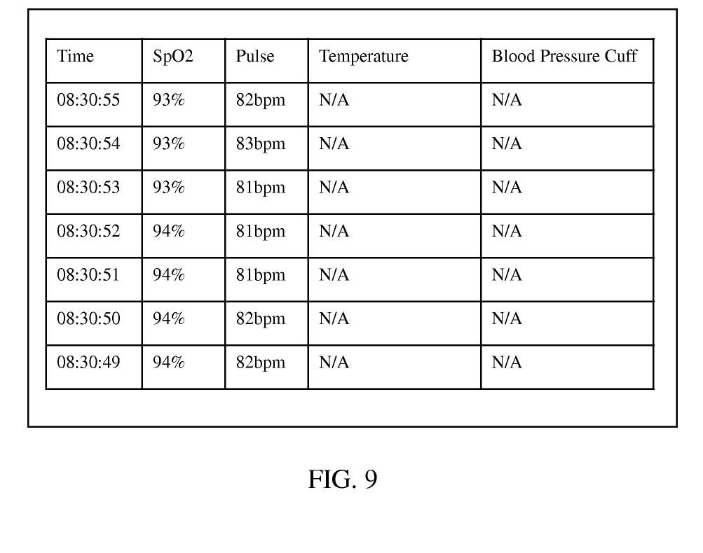

FIG. 9 shows and exemplary sensor database 136 of the health monitor 130. The sensor database 136 contains a data set that comprises time and sensor readings from the different monitoring devices connected to the health monitor 130. The monitoring devices included varies for each disease state being monitored. As an example for this sensor database 136, the data set includes blood oxygen saturation or SpO2 data, pulse rate, temperature and blood pressure. The sensor database 136 may vary according to the disease being monitored by the at least one kit.

Blood oxygen saturation is a measure of the amount of oxygen carried by hemoglobin in the blood stream. It is usually expressed as a percentage rather than an absolute reading. For example, blood oxygen saturation levels measured immediately after birth can provide a good indicator of a baby's general state of health. Levels below 75% could indicate that the newborn infant may be suffering from some abnormality. To determine a patient's condition, the blood oxygen saturation should be expressed as a percentage of the total hemoglobin that is saturated with oxygen. Under many circumstances, that percentage is the reading that pulse oximeters provides. Acceptable normal ranges for healthy patients range from 95 to 99 percent.

A typical pulse oximeter comprises an electronic processor and a couple of small light-emitting diodes (LEDs) facing a photodiode through a translucent portion of a patient's body, typically a fingertip or an earlobe. One of the LEDs emits light in the red portion of the visible region of the electromagnetic spectrum (red LED) while the other emits in the infrared region. The amount of light absorbed at these two wavelengths differs significantly between oxygen-rich blood and blood deficient in oxygen. Oxygenated hemoglobin absorbs more infrared light and allows more red light to pass through. On the other hand, deoxygenated hemoglobin allows more infrared light to pass through and absorbs more red light. Oxy-hemoglobin and its deoxygenated form have significantly different absorption pattern.

In operation, the LEDs alternately turn on and off, and then both off approximately for a predetermined period of time. This allows the light sensor, for example, a photodiode, to respond to the red and infrared light separately and also to correct for the light detected due to ambient light (measured when both LEDs are off; used as baseline or reference signal). The amount of light that is transmitted (that is, not absorbed) is measured, and separate normalized signals are produced for each wavelength. These signals due to transmitted light vary with time because the amount of arterial blood that is present increases with each heartbeat. By subtracting the minimum transmitted light from the peak transmitted light in each wavelength, the effects of other tissues and materials (e.g., venous blood, skin, bone, muscle, fat, including nail polish) can be corrected for because they normally absorb a constant amount of light over a period of time. The ratio of the measured red light to the measured infrared light is calculated by the processor. This ratio, which represents the ratio of oxygenated hemoglobin to deoxygenated hemoglobin, is then converted to a SpO2 reading by the processor.

The present invention is not intended to be restricted to the several exemplary embodiments of the invention described above. Other variations that may be envisioned by those skilled in the art are intended to fall within the disclosure.

1. A system for monitoring a patient with a specific condition comprising:
a health monitor comprising at least one of a protocols and alerts database, a devices database, a sensor database, a kit loading software, a monitoring software including a treatment GUI, and a protocol update software including a device GUI;
at least one kit comprising:
one or more monitoring devices for acquiring a patient health data;
a memory device comprising:
means to connect the memory device to the health monitor; and
at least one condition-specific software comprising one or more health-monitoring protocols and one or more alerts data; and
a health network comprising at least one disease database selected from a pneumonia database, a heart attack recovery database, a hypoglycemia database, a database for a disease, and a combination thereof.

2. The system of claim 1, wherein the memory device stores a list of the one or more monitoring devices of the at least one kit.

3. The system of claim 1, wherein the health monitor comprises each of a protocols and alerts database, a devices database, a sensor database, a kit loading software, a monitoring software including a treatment GUI, and a protocol update software including a device GUI.

4. The system of claim 1, wherein the health network accessed via a cloud network connected to the health monitor.

5. The system of claim 4, wherein the health network comprises each of a pneumonia database, a heart attack recovery database, a hypoglycemia database, a database for a disease, and a combination thereof.

6. The system of claim 1, wherein the one or more monitoring devices is selected from a thermometer, a blood pressure cuff, a pulse oximeter, and a combination thereof.

7. The system of claim 1, wherein the treatment GUI shows a disease state being monitored, the one or more monitoring devices acquiring the patient health data, the patient health data acquired by the one or more monitoring devices, and a reading rate corresponding to the one or more monitoring devices.

8. The system of claim 1, wherein the patient data is selected from a blood oxygen saturation level, a pulse rate, and a temperature, a blood pressure, and a combination thereof.

9. A method for monitoring a patient with a specific condition comprising:
polling for a connection of a memory device to a health monitor until a connection is detected;
uploading and storing a list of one or more monitoring devices to a devices database, each monitoring device acquiring health data of a patient;
uploading and storing health-monitoring protocols and alerts data from the memory device to a protocol and alerts database;
accessing a health network via a cloud network;
comparing a protocols and alerts database with at least one disease database;
detecting a difference between the protocols and alerts database, and the at least one disease database;
updating the health monitor protocols and alerts database to match the at least one disease database;
polling for a connection of the one or more monitoring device to the health monitor until the connection is detected; and
executing one or more health monitoring protocols based on alert data.

10. The method of claim 9, further comprising:
displaying a treatment GUI on the health monitor;
accessing the protocols and alerts database to determine a reading rate of the one or more monitoring devices;
displaying the reading rate on the treatment GUI;
polling the one or more monitoring devices for a reading;
storing the polled reading in the sensor database;
displaying the polled reading on the treatment GUI;
accessing a sensor database to determine a time of the most recent polled reading for each of the at least one monitoring device;
calculating a time difference by subtracting the current time from a next scheduled reading time based on the reading rate of the monitoring device;
displaying the calculated time difference on the treatment GUI;
accessing the protocols and alerts database and executing an alert action corresponding to the one or more monitoring devices; and
executing a protocol update software.

11. The method of claim 10, further comprising:
accessing the health network;
comparing at least one disease database corresponding to a kit with a most recent entry of the sensor database;
saving a matching device, a health signal, a reading rate, and alert action data on the corresponding health network database to the health monitor protocols and alerts database;
comparing the protocols and alerts database with the devices database and determining that there is a monitoring device in the protocols and alerts database that is missing from the devices database;
saving the determined missing device to the devices database;
displaying a device GUI;
displaying the determined missing device on the device GUI; and
polling for a connection of the one or more monitoring devices in the devices database until connection for all of the one or more monitoring devices in the devices database is detected.

12. The method of claim 10, wherein the treatment GUI shows a disease state being monitored, the one or more monitoring devices acquiring the patient health data, the patient health data acquired by the one or more monitoring devices, and the reading rate corresponding to the one or more monitoring devices.

13. The method of claim 9, wherein the patient data is selected from a blood oxygen saturation level, a pulse rate, and a temperature, a blood pressure, and a combination thereof.

14. The method of claim 9, wherein the health network comprises at least one disease database selected from a pneumonia database, a heart attack recovery database, a hypoglycemia database, a database for a disease, and a combination thereof.

15. The method of claim 9, wherein the one or more monitoring devices is selected from a thermometer, a blood pressure cuff, a pulse oximeter, and a combination thereof.
